(19) United States
(12) Patent Application Publication
(10) Pub. No.: US 2014/0142311 A1
(43) Pub. Date: May 22, 2014

(54) METHOD FOR PREPARING PRASUGREL

(71) Applicants: Chuangwei Liu, Linhai (CN); Qifeng Lu, Linhai (CN); Changhui Chen, Linhai (CN)

(72) Inventors: Chuangwei Liu, Linhai (CN); Qifeng Lu, Linhai (CN); Changhui Chen, Linhai (CN)

(73) Assignee: Zhejiang Huahai Pharmaceutical Co., Ltd., Zhejiang (CN)

(21) Appl. No.: 14/165,446

(22) Filed: Jan. 27, 2014

Related U.S. Application Data
(62) Division of application No. 13/383,192, filed on Jan. 9, 2012.

(30) Foreign Application Priority Data
Aug. 26, 2009 (CN) 200910170675.X

(51) Int. Cl. C07D 495/04

(52) U.S. Cl. CPC C07D 495/04
USPC 546/114

(57) ABSTRACT

The present invention relates to a method for synthesizing prasugrel, comprising the following steps: converting o-fluorobenzyl cyclopropyl ketone into α-cyclopropylcarbonyl-2-fluorobenzyl halide (compound 2) using dibromohydantoinhydantoin as halogenation reagent and acetic acid as solvent, then 2-oxo-4,5,6,7-tetrahydrothieno[3,2-c]pyridine p-toluenesulfonate (compound 4) is obtained with high yield by a concerted catalysis using a phase transfer catalyst and an inorganic salt, then is condensed and acylated to obtain prasugrel as a gum. The present invention also provides a method for purifying prasugrel comprising crystallizing using alcohols as a crystallization solvent to obtain prasugrel crystals with a high purity.

The present application is a division of U.S. patent application Ser. No. 13/383192, filed on Jan. 9, 2012, which application claims the priority of Chinese patent application No. 200910170675.X filed in the Chinese Patent Office on Aug. 26, 2009, titled “An Artificial Method for Synthesis of Prasugrel”, which applications are hereby incorporated by reference to the maximum extent allowable by law.

### FIELD OF THE INVENTION

The present invention belongs to the pharmaceutical and chemical field, in particular, relates to a preparation method of prasugrel and crystallization method thereof.

### BACKGROUND OF THE INVENTION

Prasugrel is an oral anti-platelet drug co-developed by Eli Lilly and its partner Daiichi Sankyo Co. Ltd. It has been reported that prasugrel hydrochloride and prasugrel maleate both have good anti-thrombotic activies and are a new generation of potent thienopyridines anti-platelet drug, and thus much attention has been paid to the synthesis of prasugrel compounds. The structural formula of prasugrel is as follows:

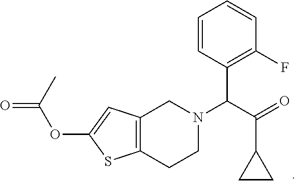 [SMILES: CC(=O)Oc1cc2c(s1)CCN(C(C(=O)C1CC1)c1ccccc1F)C2]

Several methods for synthesizing prasugrel in the form of free base were reported in European Patent No. 0542411, US Patent No. 20030134872 and European Patent No.0785205. All of these methods comprise firstly condensating α-cyclopropylcarbonyl-2-fluorobenzyl halide (compound 2) with 2-oxo-4,5,6,7-tetrahydrothieno [3 ,2-c]pyridine salt (compound 3) to obtain 5-(α-cyclopropylcarbonyl-2-fluorobenzyl)- 2-oxo-2,4,5 ,6,7-tetrahydrothieno [3,2-c]pyridine (compound 4), and compound 4 is finally acylated and purified to give prasugrel (compound 5).

However, the processes provided in the above patents have the following defects: (1) the yield for the synthetic step of intermediate compound 4 is low (only 32% to 35%); (2) the cost of the brominating agent NBS adopted is high, moreover, the toxicity of the solvents used such as carbon tetrachloride or chloroform is relatively high, and are not good for the environmental protection; and (3) the target compound is obtained by final purification through column chromatography, as a result, the separation method is complicated and the cost of separation is high. Thus, these processes are not suitable for large-scale industrial production.

### SUMMARY OF THE INVENTION

The purpose of the present invention is to provide a synthesis method which is safe, economic and easy to operate and purify, and has a high yield, thus is suitable for large-scale industrial production.

To achieve this purpose, the technical solutions used in the present invention are as follows:

A crystalline form of prasugrel crystal powder, characterized in that the X-ray polycrystalline diffraction pattern of prasugrel crystal powder has the following characteristic diffraction peaks (2θ): 7.63±0.2, 11.11±0.2, 13.34±0.2, 14.63±0.2, 18.48±0.2, 18.76±0.2, 19.22±0.2, 20.64±0.2, 21.30±0.2, 22.64±0.2, 23.30±0.2, 24.68±0.2, 26.22±0.2, 26.86±0.2, 29.42±0.2 and 31.28±0.2.

The melting point of the crystalline form as mentioned above is 121.3° C.-125.2° C.

A method for synthesizing crystalline form A of prasugrel, comprising the following steps:

(1) o-fluorobenzyl cyclopropyl ketone as a raw material is reacted with 1,3-dibromo-5,5-dimethylhydantoin (DBDMH) or 1,3-dichloro-5,5-dimethylhydantoin as a halogenating agent in acetic acid as a solvent at a temperature of 0° C.-100° C. to give α-cyclopropylcarbonyl-2-fluorobenzyl halide (compound 2).

Preferably, at a temperature of 60° C.-85° C., α-cyclopropylcarbonyl-2-fluorobenzyl halide (compound 2) is mildly formed using o-fluorobenzyl cyclopropyl ketone as raw material, 1,3-dibromo-5,5-dimethylhydantoin or 1,3-dichloro-5,5-dimethylhydantoin as a halogenating agent, and acetic acid as a solvent by addition in dropwise, thus the outbreak of free radical reaction is avoided. The molar ratio of O-fluorobenzyl cyclopropyl ketone to the halogenating agent is 1:0.5-0.65, preferably 1:0.6. Compared with the process used in U.S. Pat. No. 20030134872, the operating conditions are greatly improved by using 1,3-dibromo-5,5-dimethylhydantoin as a halogenating agent, and the selectivity and efficiency of halogenation are greatly improved. Environmental pollution is reduced and the degree of environmental protection is increased by using acetic acid as a solvent compared to the process using chloroform.

(2) α-cyclopropylcarbonyl-2-fluorobenzyl halide (compound 2) is then reacted with 2-oxo-4,5,6,7-tetrahydrothieno[3,2-c]pyridine p-toluenesulfonate (compound 3) in the concerted catalysis of a phase transfer catalyst and an inorganic salt using an inorganic base as an acid binding agent and DMF as a solvent under nitrogen atmosphere to produce 5-(α-cyclopropylcarbonyl-2-fluorobenzyl)-2-oxo-2,4,5,6,7-tetrahydrothieno[3,2-c]pyridine (compound 4).

In this step, the molar ratio of compound 2 to compound 3 is 1:1-1.5, preferably 1:1.15. The molar ratio of compound 2 to the inorganic alkali is 1:2-8, preferably 1:4. The inorganic alkali can be one or more selected from the group consisting of sodium carbonate, potassium carbonate, sodium bicarbonate and potassium bicarbonate, more preferably sodium bicarbonate. The molar ratio of compound 2 to the phase transfer catalyst is 1:0.05-1.2, preferably 1:1.01. The phase transfer catalyst can be selected from quaternary ammonium salts, such as tetrabutyl ammonium bromide, tetraethyl ammonium bromide, tetrabutyl ammonium chloride, tetraethyl ammonium chloride and the like. The molar ratio of compound 2 to the inorganic salt is 1:0.02-1.2, preferably 1:0.1. The inorganic salt includes bromides, such as sodium bromide and potassium bromide.

In the above step, the temperature of the catalytic reaction is usually 0° C.-100° C., preferably 40° C.-50° C.

A catalytic amount of a phase transfer catalyst is used in combination with an inorganic salt to perform a concerted catalysis in this step. Inorganic salts such as sodium bromide may have a catalytic or salt effect on the nucleophilic substitution reaction in this reaction process. Compared with the process described in U.S. Pat. No. 20030134872, the reaction yield is improved to 90% and the reaction time is shortened to 45 minutes.

(3) Compound 4 is acylated in the presence of an acylating agent using triethylamine as an acid binding agent and DMF as a solvent under nitrogen atmosphere at room temperature, and finally extracted, dried and filtrated to give the mother liquor of prasugrel. The molar ratio of compound 4 to triethylamine in this step is 1:1-10, preferably 1:3. The acylating agent used can be selected from acetic anhydride or acetyl chloride, preferably acetic anhydride.

(4) An alcoholic solvent is added to the prasugrel gum obtained in the above step, a crystal is precipitated after stirring at room temperature. The crystal is filtrated to give prasugrel solid. The alcoholic solvents include one or more selected from the group consisting of aliphatic alcohols such as methanol, ethanol, isopropanol, n-butanol, tert-butanol, benzyl alcohol and aromatic alcohols such as benzyl alcohol. The alcoholic solvent is preferably methanol, ethanol and isopropyl alcohol.

The ratio of the volume of the alcoholic solvent used to the weight of α-cyclopropylcarbonyl-2-fluorobenzyl halide is 2.5ml/g-6.5ml/g, preferably 3.0ml/g.

The purity of prasugrel obtained in this step is ≧95%. The product is recrystallized once again from ethanol to get a white crystal whose purity is ≧99.50%.

In this step, an alcoholic solvent as the crystallization and recrystallization solvent is used for the final separation and purification of prasugrel, to give the title compound by crystallize and purification. The separation method of the present invention is more simple, feasible, economic and favorable for large-scale industrial production compared with the separation and purification process (column chromatography) in U.S. Pat. No. 20030134872.

The reaction scheme of the invention is as follows:

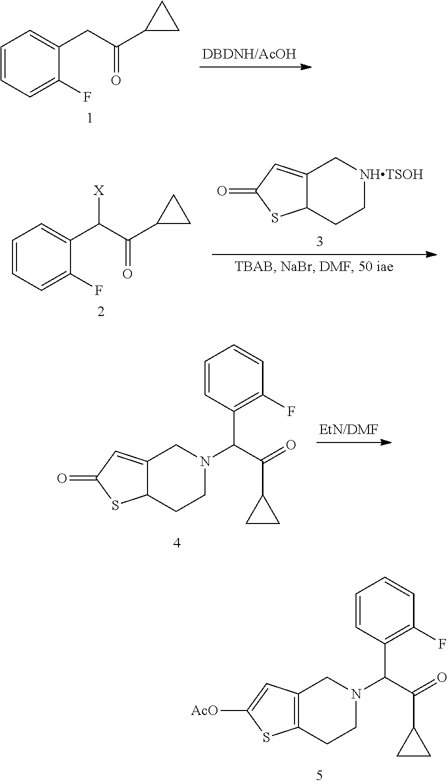

The synthesis method for prasugrel provided in the present invention employs an alcoholic solvent as the crystallization and recrystallization solvent for crystallization and purification and the yield of the reaction is high. The method has the advantages of safety, economy, low-pollution and simple operation, thus, it is suitable for large-scale industrial production.

### BRIEF DESCRIPTION OF THE DRAWINGS

FIG. 1 is the X-ray powder diffraction pattern of crystalline form A of prasugrel powder crystal. The lateral axis represents the diffraction angle (2θ), and the vertical axis represents intensity (CPS).

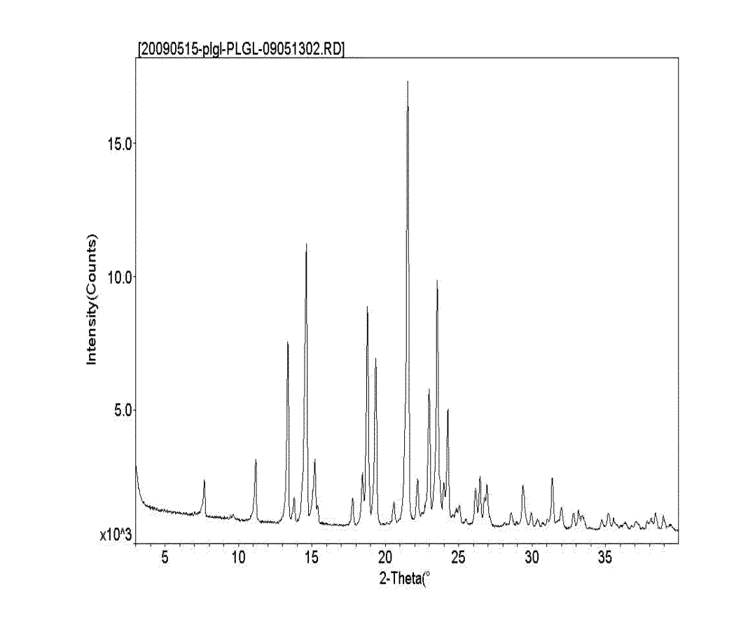

### DETAILED DESCRIPTION OF THE INVENTION

Now the present invention will be further illustrated in combination with the following examples, so that those skilled in the art can understand the present invention better. However, the scope of the present invention is not limited in any way.

### Example 1

Preparation of α-cyclopropylcarbonyl-2-fluorobenzyl Bromide (Compound 2):

13.1 g o-fluorobenzyl cyclopropyl ketone and 40 ml acetic acid were added to a 100 ml four-neck flask equipped with a mechanical stirring device, a thermometer, a reflux condenser and a constant-pressure dropping funnel. 12.1 g 1,3-dibromo-5,5-dimethylhydantoin and 0.66 g azobisisobutyronitrile were added in dropwise to the above reaction system at 60° C.-85° C. over 2 hours. After the completion of addition, the mixture was kept at this temperature and stirred for 25 minutes. Next, the mixture was cooled and distilled to remove most of acetic acid. 40ml ethyl acetate and 40 ml water were added to the concentrated solution, and the system was allowed to separation. The organic layer was washed with 20 ml saturated Na₂SO₃, 20 ml saturated NaHCO₃ and 20 ml saturated brine successively, and then dried over anhydrous magnesium sulfate. The filtrate was distillated under reduced pressure and concentrated to give 21.1 g brown oil. The yield was 83.5% and the purity was 74.8%.

### Example 2

Preparation of α-cyclopropylcarbonyl-2-fluorobenzyl Chloride (Compound 2):

13.1 g o-fluorobenzyl cyclopropyl ketone and 40 ml acetic acid were added to a 100 ml four-neck flask equipped with a mechanical stirring device, a thermometer, a reflux condenser and a constant-pressure dropping funnel. 9.9 g 1,3-dibromo-5,5-dimethylhydantoin was added in dropwise to the above reaction system over 0.5 hour. After the completion of addition, the mixture was stirred for 2 hours at room temperature. Next, the mixture was cooled and distilled to remove most of acetic acid. 40 ml ethyl acetate and 40 ml water were added to the concentrated solution, and the system was allowed to separation. The organic layer was washed with 20 ml saturated Na₂SO₃, 20 ml saturated NaHCO₃ and 20 ml saturated brine successively, and then dried over anhydrous magnesium sulfate. The filtrate was distillated under reduced pressure and concentrated to give 16.6 g brown oil. The yield was 79.5% and the purity was 75.0%.

### Example 3

Preparation of 5-(α-cyclopropylcarbonyl-2-fluorobenzyl)-2-oxo -4,5,6,7-tetrahydrothieno [3,2-c]pyridine (Compound 4)

25.7 g α-cyclopropylcarbonyl-2-fluorobenzyl bromide prepared in Example 1, 37.6 g 2-oxo-4,5,6,7-tetrahydrothieno[3,2-c]pyridine p-toluenesulfonate, 33.6 g sodium bicarbonate, 1.0 g sodium bromide, 3.2 g tetrabutylammonium bromide and 150 ml DMF were added to a 1000 ml four-neck flask equipped with a mechanical stirring device, a thermometer, a reflux condenser and a constant-pressure dropping funnel. The mixture was stirred for 45 minutes under nitrogen atmosphere at 40° C.-55° C. and cooled. 200ml ethyl acetate and 300 ml water were added thereto, and the system was allowed to separation. The organic layer was washed with 300 ml water and 100 ml saturated brine successively, and then dried over anhydrous magnesium sulfate. The resulting mixture was decolored with silica gel bed and filtered. The filtrate was distillated under reduced pressure and concentrated to give 26.75 g brown gum. The yield was 94.5% and the purity was 87.46%.

### Example 4

Preparation of 5-(α-cyclopropylcarbonyl-2-fluorobenzyl)-2-oxo -4,5,6,7-tetrahydrothieno [3,2-c]pyridine (Compound 4)

25.7 g α-cyclopropylcarbonyl-2-fluorobenzyl bromide prepared in Example 1, 37.6 g 2-oxo-4,5,6,7-tetrahydrothieno[3,2-c]pyridine p-toluenesulfonate, 40.0 g potassium bicarbonate, 8.24 g sodium bromide, 3.2 g tetrabutylammonium bromide and 150 ml DMF were added to a 1000 ml four-neck flask equipped with a mechanical stirring device, a thermometer, a reflux condenser and a constant-pressure dropping funnel. The mixture was stirred for 45 minutes under nitrogen atmosphere at 40° C.-55° C. and cooled. 200 ml ethyl acetate and 300 ml water were added thereto, and the system was allowed to separation. The organic layer was washed with 300 ml water and 100 ml saturated brine successively, and then dried over anhydrous magnesium sulfate. The resulting mixture was decolored with silica gel bed and filtered. The filtrate was distillated under reduced pressure and concentrated to give 27.10 g brown gum. The yield was 91.0% and the purity was 83.21%.

### Example 5

Preparation of 5-(α-cyclopropylcarbonyl-2-fluorobenzyl)-2-oxo -4,5,6,7-tetrahydrothieno [3,2-c]pyridine (Compound 4)

25.7 g α-cyclopropylcarbonyl-2-fluorobenzyl chloride prepared in Example 2, 47.5 g 2-oxo-4,5,6,7-tetrahydrothieno[3,2-c]pyridine p-toluenesulfonate, 40.6 g sodium bicarbonate, 0.95 g potassium bromide, 2.6 g tetrabutylammonium bromide and 180 ml DMF were added to a 1000 ml four-neck flask equipped with a mechanical stirring device, a thermometer, a reflux condenser and a constant-pressure dropping funnel. The mixture was stirred for 45 minutes under nitrogen atmosphere at 40° C.-55° C. and cooled. 220 ml ethyl acetate and 350 ml water were added thereto, and the system was allowed to separation. The organic layer was washed with 350 ml water and 120 ml saturated brine successively, and then dried over anhydrous magnesium sulfate. The resulting mixture was decolored with silica gel bed and filtered. The filtrate was distillated under reduced pressure and concentrated to give 38.45 g brown gum. The yield was 72.3% and the purity was 86.45%.

### Example 6

Preparation of 2-acetyloxy-5-(α-cyclopropylcarbonyl-2-fluorobenzyl)-4,5,6,7-tetrahydrothieno[3,2-c]pyridine (Prasugrel)

18.76 g 5-(α-cyclopropylcarbonyl-2-fluorobenzyl)-2-oxo-4,5,6,7-tetrahydrothieno[3,2-c]pyridine (compound 4) prepared in Example 3, 4 or 5 and 60 ml DMF were added to a 500 ml four-neck flask equipped with a mechanical stirring device, a thermometer and a constant-pressure dropping funnel. The flask was placed in a water-ice bath and 17.17 g triethylamine was injected under nitrogen atmosphere. The temperature was controlled at −10° C. to 5° C. and 17.34 g acetic anhydride was added slowly in dropwise over 30 minutes. After the completion of addition, the mixture was kept for 10 minutes at this temperature and then stirred for 2 hours at room temperature. 100 ml ethyl acetate and 150 ml water were added thereto, and the system was allowed to separation. The organic layer was washed with 150 ml water, 50 ml saturated NaHCO₃ and 50 ml saturated brine successively, and then dried over anhydrous magnesium sulfate. The resulting mixture was decolored with silica gel bed and filtered. The filtrate was distillated under reduced pressure and concentrated to give 26.10 g brown gum.

### Example 7

26.10 g prasugrel gum prepared in Example 6 was added to 70 ml ethanol. The mixture was stirred for 12 hours at room temperature under nitrogen atmosphere, crystallized and filtered to give 12.2 g off-white solid. The yield was 56.0% and the purity was 97.02%. The product was recrystallized from 33 ml ethanol to give 10.6 g white solid. The melting point was 123° C. and the purity was 99.75%.

### Example 8

26.1 g prasugrel gum prepared in Example 6 was added to 100 ml isopropanol. The mixture was stirred for 12 hours at room temperature under nitrogen atmosphere, crystallized and filtered to give 10.5 g off-white solid. The yield was 48.2% and the purity was 99.54%. The product was recrystallized from 42 ml isopropanol to give 9.0 g white solid. The melting point was 122° C. and the purity was 99.80%.

The present invention has been described in detail hereinbefore, including its preferred embodiments. However, it should be appreciated that in consideration of the contents disclosed in the present invention, modifications and/or improvements may be made on the present invention by those skilled in the art within the spirit of the claims of the invention. These modifications and/or improvements should also be deemed to fall within the protection scope of the present invention.

What is claimed is:

1. A method for synthesizing crystalline form A of prasugrel, wherein the crystalline form A has the following X-ray powder diffraction pattern measured by copper cathode diffractometer, expressed as the interplanar spacing d, Bragg angle 2θ, intensity and relative intensity, wherein the relative intensity is expressed as a percentage relative to the strongest ray:

| | interplanar spacing | | relative |
|---|---|---|---|
| 2θ angle (°) | d(Å) | intensity | intensity (%) |
| 7.639 | 11.56 | 1569 | 19 |
| 9.717 | 9.09 | 192 | 2.3 |
| 11.118 | 7.95 | 2838 | 34.4 |
| 13.340 | 6.63 | 8253 | 100 |
| 13.679 | 6.47 | 808 | 9.8 |
| 14.360 | 6.16 | 4184 | 50.7 |
| 14.639 | 6.05 | 5807 | 70.4 |
| 14.980 | 5.91 | 2125 | 25.7 |
| 17.737 | 5.00 | 720 | 8.7 |
| 18.480 | 4.80 | 2087 | 25.3 |
| 18.761 | 4.73 | 8089 | 98 |
| 19.221 | 4.61 | 6421 | 77.8 |
| 20.640 | 4.30 | 707 | 8.6 |
| 21.301 | 4.17 | 7910 | 95.8 |
| 22.298 | 3.98 | 1681 | 20.4 |
| 22.640 | 3.92 | 1743 | 21.1 |
| 23.300 | 3.81 | 7209 | 87.4 |
| 24.258 | 3.67 | 6855 | 83.1 |
| 24.682 | 3.60 | 649 | 7.9 |
| 26.221 | 3.40 | 1237 | 15 |
| 26.862 | 3.32 | 1947 | 23.6 |
| 28.320 | 3.15 | 501 | 6.1 |
| 29.422 | 3.03 | 548 | 6.6 |
| 30.179 | 2.96 | 323 | 3.9 |
| 31.280 | 2.86 | 2091 | 25.3 |
| 31.922 | 2.80 | 632 | 7.7 |
| 32.780 | 2.73 | 924 | 11.2 |
| 33.421 | 2.68 | 362 | 4.4 |
| 34.960 | 2.56 | 531 | 6.4 |
| 35.557 | 2.52 | 218 | 2.6 |
| 36.960 | 2.43 | 486 | 5.9 |
| 38.060 | 2.36 | 583 | 7.1 |
| 38.339 | 2.35 | 358 | 4.3 |
| 39.000 | 2.31 | 953 | 11.5 |

characterized in that the method comprises the following steps:
(1) o-fluorobenzyl cyclopropyl ketone as a raw material is reacted with 1,3-dibromo-5,5-dimethylhydantoin or 1,3-dichloro-5,5- dimethylhydantoin as a halogenating agent in acetic acid as a solvent to give α-cyclopropylcarbonyl-2-fluorobenzyl halide (compound 2);
(2) α-cyclopropylcarbonyl-2-fluorobenzyl halide is reacted with 2-oxo-4,5,6,7-tetrahydrothieno[3,2-c]pyridine toluenesulfonate (compound 3) in the concerted catalysis of a phase transfer catalyst and an inorganic salt using an inorganic base as an acid binding agent and DMF as a solvent under nitrogen atmosphere to get 5-(α-cyclopropylcarbonyl-2-fluorobenzyl)-2-oxo-2,4,5,6,7-tetrahydrothieno [3,2-c]pyridine (compound 4);
(3) 5-(α-cyclopropylcarbonyl-2-fluorobenzyl)-2-oxo-2,4,5,6,7-tetrahydrothieno[3,2-c]pyridine is acylated in the presence of an acylating reagent using triethylamine as an acid binding agent and DMF as a solvent under nitrogen atmosphere at room temperature, and finally extracted, dried and filtrated to give the mother liquor of prasugrel, and the mother liquor is concentrated to get prasugrel as a gum;
(4) an alcoholic solvent is added to the prasugrel as a gum obtained in the above step, and crystal is precipitated after stirring at room temperature, then the crystal is filtrated to give prasugrel as solid;
the reaction scheme is as follows:

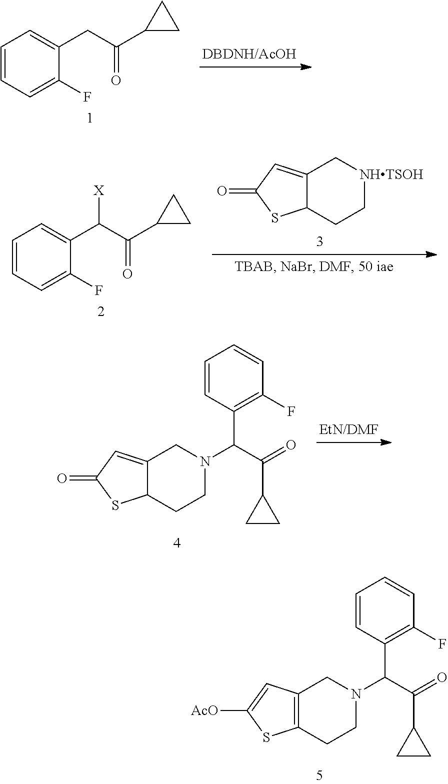

2. The synthesis method according to claim 1, characterized in that the reaction temperature in step (1) is 0° C. to 100° C.

3. The synthesis method according to claim 2, characterized in that the reaction temperature is 60° C. to 85° C.

4. The synthesis method according to claim 1, characterized in that the molar ratio of o-fluorobenzyl cyclopropyl ketone to the halogenating agent is 1:0.5-0.65.

5. The synthesis method according to claim 1, characterized in that the molar ratio of α-cyclopropylcarbonyl-2-fluorobenzyl bromide to 2-oxo-4,5,6,7-tetrahydrothieno[3,2-c]pyridine p-toluenesulfonate in step (2) is 1:1-1.5.

6. The synthesis method according to claim 1, characterized in that the molar ratio of α-cyclopropylcarbonyl-2-fluorobenzyl bromide to the inorganic base in step (2) is 1:2-8.

7. The synthesis method according to claim 1, characterized in that the inorganic base is one or more selected from the group consisting of sodium carbonate, potassium carbonate, sodium bicarbonate, and potassium bicarbonate.

8. The synthesis method according to claim 7, characterized in that the inorganic base is sodium bicarbonate.

9. The synthesis method according to claim 1, characterized in that the molar ratio of α-cyclopropylcarbonyl-2-fluorobenzyl bromide to the phase transfer catalyst in step (2) is 1:0.05-1.2.

10. The synthesis method according to claim 1, characterized in that the phase transfer catalyst comprises one or more selected from the group consisting of tetrabutylammonium bromide, tetraethyl ammonium bromide, tetrabutyl ammonium chloride and tetraethylammonium chloride.

11. The synthesis method according to claim 1, characterized in that the molar ratio of α-cyclopropylcarbonyl-2-fluorobenzyl bromide to the inorganic salt in step (2) is 1:0.02-1.2.

12. The synthesis method according to claim 1, characterized in that the inorganic salt comprises one or more selected from the group consisting of sodium bromide and potassium bromide.

13. The synthesis method according to claim 1, characterized in that the reaction temperature in step (2) is 0° C.-100° C.

14. The synthesis method according to claim 13, characterized in that the temperature of the reaction is 40° C.-50° C.

15. The synthesis method according to claim 1, characterized in that the molar ratio of 5-(α-cyclopropylcarbonyl-2-fluorobenzyl)-2-oxo-2,4,5,6,7 -tetrahydrothieno [3 ,2-c]pyridine to triethylamine in step (3) is 1:1-10.

16. The synthesis method according to claim 1, characterized in that the alcoholic solvent in step (4) comprises one or more selected from the group consisting of methanol, ethanol, isopropanol, n-butanol, tert-butanol, benzyl alcohol or benzoic alcohol.

17. The synthesis method according to claim 16, characterized in that the alcoholic solvent is methanol, ethanol or isopropanol.

18. The synthesis method according to claim 1, characterized in that the ratio of the volume of the alcoholic solvent to the weight of α-cyclopropylcarbonyl-2-fluorobenzyl halide is 2.5 ml/g-6.5 ml/g.

19. The synthesis method according to claim 18, characterized in that the ratio of the volume of the alcoholic solvent to the weight of α-cyclopropylcarbonyl-2-fluorobenzyl halide is 3.0 ml/g.
